(19) United States
(12) Patent Application Publication
(10) Pub. No.: US 2004/0219193 A1
(43) Pub. Date: Nov. 4, 2004

(54) Adhesive composition and low temperature applicable adhesive sheet

(76) Inventors: Hidetoshi Abe, Yamagata (JP); Yorinobu Takamatsu, Kanagawa (JP)

Correspondence Address: 3M INNOVATIVE PROPERTIES COMPANY, PO BOX 33427, ST. PAUL, MN, 55133-3427, US

(21) Appl. No.: 10/487,369

(22) PCT Filed: Aug. 27, 2002
(86) PCT No.: PCT/US02/27334

(30) Foreign Application Priority Data
Aug. 31, 2001 (JP) 2001-262609

(51) Int. Cl. A61K 9/70

(52) U.S. Cl. 424/449

(57) ABSTRACT

An adhesive composition having pressure sensitivity includes an adhesive polymer and a nonadhesive polymer. The adhesive polymer has a glass transition point by a dynamic viscoelasticity measuring method of −60° C. to −5° C., and the nonadhesive polymer has a glass transition point by a differential scanning calorimeter of −5° C. or less, and a fusing point of higher than 25° C. by a differential scanning calorimeter, and also has compatibility with the adhesive polymer in uncrystallized state. The adhesive composition can effectively enhance pressure sensitive adhesivity at low temperatures and tack suppression effect at the same time, which makes easy an accurate positioning of the site for sticking, and which can be easily stuck by press fitting even in an environment of low temperature of lower than 0° C.

### BACKGROUND OF THE INVENTION

1. Technical Field

The present invention relates to an adhesive composition and an adhesive sheet, and in more detail to an adhesive composition having pressure sensitivity including an adhesive polymer and a nonadhesive polymer, and to an adhesive sheet.

2. Related Art

An adhesive sheet equipped with an adhesive layer comprising a pressure sensitive adhesives on at least one of main surfaces of a substrate (surface side or back side) may be easily and strongly stuck only by pressure being applied, and thus since sticking work to adherend can be easily done manually, these adhesive sheets are used widely.

Conventionally, as pressure sensitive adhesives constituting adhesive layers of adhesive sheets, a pressure sensitive adhesive having adhesive polymers as principal component is widely known, and various types have been developed.

For example, a pressure sensitive adhesives including a) acrylate copolymer 100 mass parts obtained from a monomer mixture including one or more kinds of mono functional acrylates having non-tertiary alkyl group having 1 to 14 carbons about 70 to 98 percent by weight, and a polar monomer of approximately 30 to 2 percent by weight, b) a plasticizer of 2 to 10 mass parts are known.

Typically, pressure sensitive adhesives, wherein acrylate copolymers included as monomer units (repetition units) originating in mono functional acrylates at comparatively large percentage in molecule, almost all copolymers show a glass transition point (Tg) by a dynamic viscoelasticity measuring method of −5° C. or less. Furthermore plasticizers, such as polyethylene oxides and adipates, may also be added, the pressure sensitive adhesives are excellent in low-temperature performance as low-temperature adhesive property. For the above reason when adhesive layers arranged on back side of an adhesive sheet that is used out in the fields for advertising object, such as graphics display sheets, are constituted with this pressure sensitive adhesives, even in the case where they are used in a low-temperature environment as at 20° F. (−7° C.), sufficient adhesive property (releasing strength and holding power) is realized.

However, because plasticizers usually have low crystallinity, tack (hereinafter, referred to as a property with which sticking is carried out with slight power) of an adhesive layer may not be controlled. Therefore, even when an adhesive coated article was stuck to adherend surface lightly, adhesive sheet could not be released easily after adhesion, so that there occurred a problem that difficulty was induced in accurate positioning of site for sticking.

On the other hand, adhesive films are known wherein tack of adhesive layers is suppressed. For example, an adhesive film in which good positioning property at ordinary temperature is realized by covering a part of the adhesive layer surface by non-tacky grains, such as inorganic grains and glass beads.

A pressure sensitive adhesive is known in which a nonadhesive polymer comprising substantially linear polycaprolactones having a molecular weight of approximately 3,000 to approximately 342,000 etc. is included in addition to pressure sensitive adhesive basic resin comprising adhesive polymers, such as acrylic derived polymers, and non-tacky grains comprising inorganic grains, such as silica, is included to mitigate tack at ordinary temperature effectively and to improve positioning property in sticking process.

An adhesive composition with thermo sensitive adhesiveness is known in which an adhesive polymer having two functional groups, hydroxy group and phenyl group, in a molecule, and crystalline polycaprolactone serving as a nonadhesive polymer are included. Compatibility between the adhesive polymer and the polycaprolactone are improved and tack on a surface of the adhesive at ordinary temperature is effectively mitigable.

However, in these conventional adhesive compositions, although adhesive films constituted with these adhesive layers were generally used outdoors for an object, such as advertisement, pressure sensitivity under low-temperature conditions was not necessarily enough.

Namely, in these conventional adhesive compositions, since nothing was taken into consideration regarding pressure sensitivity at low-temperature (especially low temperature of less than 0° C.) conditions, when tack at ordinary temperature is suppressed a tendency was observed that low-temperature adhesive property is decreased, and in some case there occurred a problem that little pressure sensitivity is demonstrated under low-temperature conditions as whole composition.

### SUMMARY OF THE INVENTION

Briefly, the present invention provides an adhesive composition that effectively enhances pressure sensitive adhesivity at low temperatures and tack suppression effect at the same time, which makes easy an accurate positioning of the site for sticking, and which can be easily stuck by press fitting even in an environment of low temperature less than 0° C., and at providing an adhesive sheet constituted with the composition as an adhesive layer.

Advantageously, when an adhesive composition is constituted with a adhesive polymer and a nonadhesive polymer, viscoelasticity behavior of the adhesive polymer has direct influence over pressure sensitivity required for adhesive composition, and that selection of a composition having a glass transition point obtained from viscoelasticity behavior in a specific range demonstrates appropriate pressure sensitivity even at low temperature.

Moreover, in a nonadhesive polymer, it was found that a degree of restraint of molecular motion resulting from crystalline grade of a polymer directly affected tack suppression effect required for adhesive composition, and that appropriate tack suppression effect was demonstrated even at low temperature by selecting a composition having a glass transition point obtained from degree (directly affected by thermal property of polymer) of restraint of this molecular motion in a specific range. And furthermore, it was found out that coexistence of the both polymer was enabled, without spoiling these performances, if the nonadhesive polymer had compatibility with the adhesive polymer in uncrystallized state, and thus the present invention was attained.

According to present invention, an adhesive composition is provided having pressure sensitivity including an adhesive polymer and a nonadhesive polymer, wherein the adhesive polymer has a glass transition point by a dynamic viscoelasticity measuring method of −60° C. to −5° C., and the nonadhesive polymer has a glass transition point by a differential scanning calorimeter of −5° C. or less, and a fusing point of higher than 25° C. by a differential scanning calorimeter, and also has compatibility with the adhesive polymer in uncrystallized state.

A first solution in which the above described adhesive polymer is dissolved, and a second solution in which the above described nonadhesive polymer is dissolved are mixed almost uniformly, and then resulting mixed solution is dried to give an adhesive composition of the present invention. Preferrably, an adhesive composition of the present invention has a probe tack lower than 7N, measured by a method based on ASTM D2979 under conditions of 25° C., contact pressure of 100 g/cm², contact time of 1 second, and releasing speed of 10 mm/second is lower than 7N, and a 180 degrees releasing strength measured at a releasing speed of 300 mm/minute, in −5° C. environment is 20 N/25 mm or more, after being stuck to a melamine baking finish plate and being left to stand for 5 minutes under −5° C. environment.

Moreover, in the present invention an adhesive polymer preferably has at least one alkyl group having 4 to 8 carbons including at least one butyl group, and at least one carboxyl group, and monomer units including alkyl groups having 4 to 8 carbons in a percentage of 60 to 99 mole % in all molecules.

And in the present invention an adhesive polymer preferably has a polycaprolactone skeleton or a polycarbonate skeleton in the molecule.

A method for manufacturing an adhesive composition is also provided, wherein the method comprises the steps of: (a) preparing a first solution by dissolving an adhesive polymer whose glass transition point by a dynamic viscoelasticity measuring method is −60 to −5° C. in a solvent, (b) preparing a second solution by dissolving in a solvent a nonadhesive polymer in which a glass transition point by a differential scanning calorimeter is −5° C. or less, a fusing point by a differential scanning calorimeter is higher than 25° C., and the nonadhesive polymer being compatible with the adhesive polymer in uncrystallized state, (c) mixing the first solution and the second solution approximately uniformly, and (d) drying an obtained mixed solution.

According to the present invention, a low temperature applicable adhesive sheet comprising a substrate, and an adhesive layer comprising the above described adhesive composition arranged at least on one main surface of the substrate is provided.

In a low temperature applicable adhesive sheet of the present invention, it is preferable that the substrate includes a polymer film, and has a breaking elongation of 50% or more at −5° C. at an elastic stress rate of 300 mm/min and a 50% elongation stress of 10 to 200 MPa at an elastic stress rate of 300 mm/min at −5° C.

### DETAILED DESCRIPTION OF PREFERRED EMBODIMENT

Hereinafter, embodiments of the present invention will be described concretely.

1. Adhesive Composition

An adhesive composition of the present invention is characterized by that the composition includes an adhesive polymer and a nonadhesive polymer, a glass transition point of the adhesive polymer by a dynamic viscoelasticity measuring method is −60 to −5° C., a glass transition point of the nonadhesive polymer by a differential scanning calorimeter is −5° C. or less, a fusing point by a differential scanning calorimeter is higher than 25° C., and also has compatibility with the adhesive polymer in uncrystallized state.

Thereby an adhesive sheet with an adhesive layer constituted by the above described composition concerned may easily be released after stuck to adherend face lightly to enable an easy accurate positioning to a site for sticking, and at the same time may easily be stuck by being stuck by press fitting towards an adherend face in an environment of comparatively wide temperature range (a range of −5 to 25° C. included) including low temperatures of 0 or less ° C.

In addition, although in the present invention an adhesive polymer and a nonadhesive polymer are specified by glass transition points based on different basis, respectively, a reason is that in an adhesive composition of the present invention, pressure sensitivity is appropriately reflected by a glass transition point based on viscoelasticity behavior of an adhesive polymer, and tack suppression effect is appropriately reflected by a glass transition point based on thermal property of a nonadhesive polymer according to a knowledge of the present inventors. Hereinafter, practical description will be given.

(1) Adhesive Polymer

As mentioned above, a glass transition point (Tg₁) of an adhesive polymer in the present invention by a dynamic viscoelasticity measuring method is −60 to −5° C.

If a glass transition point (Tg₁) of an adhesive polymer by a dynamic viscoelasticity measuring method exceeds −5° C., low-temperature adhesive property, especially pressure sensitivity at temperature of less than 0° C. will fall, and on the other hand tack effect cannot be effectively suppressed if a glass transition point (Tg₁) by a dynamic viscoelasticity measuring method is less than −60° C., and as a result after being stuck toward adherend face lightly, it does not release easily to make accurate positioning of site for sticking difficult.

Adhesive polymers whose glass transition point (Tg₁) by a dynamic viscoelasticity measuring method is −60 to −5° C. involved, for example, polymers, such as acrylic derived polymes, nitrile-butadiene derived copolymers (NBR etc.), styrene-butadiene derived copolymers (SBR etc.), uncrystallized polyurethanes, and silicone derived polymers, or polymers with two or more of the above described kinds mixed.

As an adhesive polymer of the present invention, a polymer having one or more alkyl groups having 4 to 8 carbons including at least one butyl groups, and one or more carboxyl groups is preferable among polymers having the above described characteristics.

Since an adhesive polymer having such substituents has a very high compatibility with a nonadhesive polymers at temperatures higher than fusing point of the nonadhesive polymers, and thus phenyl group is not needed to increase compatibility with nonadhesive polymer, a glass transition point (Tg₁) by a dynamic viscoelasticity measuring method is easily controlled in the above described range.

And, in the present invention, in order for a glass transition point (Tg₂) of an adhesive polymer to be controlled very easily in a range of −5° C. or less, it is preferable that many butyl groups are included in all alkyl groups having 4 to 8 carbons, and specifically, it is preferable that 50 to 100 mol % in alkyl group having 4 to 8 carbons are butyl groups.

Furthermore, as an adhesive polymer in the present invention, monomer units including alkyl group having 4 to 8 carbons is preferably include 60 to 99 mol % in all of molecules, more preferably 65 to 98 mol % in all of the molecules, and still more preferably 70 to 95 mol % in all of the molecules.

In the case where a percentage of the monomer unit including alkyl group having 4 to 8 carbons is less than 60 mol %, low-temperature adhesive property of the adhesive composition may not be effectively raised. On the other hand, when a content of the monomer unit including alkyl group having 4 to 8 carbons exceeds 99 mol %, a content percentage of a monomer unit including active hydrogen containing group may be decreased, and so that compatibility between the nonadhesive polymer and the adhesive polymer may be decreased.

Although there is no specific limitation about a molecular weight, if an adhesive polymer used in the present invention is in a range where predetermined adhesive strength is demonstrated, usually it may be set to have a weight average molecular weight of 10,000 to 1,000,000. As used in this application “weight average molecular weight” means a styrene converted molecular weight by a GPC method.

A cross-linked polymer may be used as an adhesive polymer to increase heat resistance, and to prevent adhesive deposit.

Cross linkage may be formed through functional groups, such as hydroxyl groups other than alkyl group and carboxyl group in the adhesive polymer, epoxy groups, and radiation-induced cross linking property functional groups, and may be formed through carboxyl groups.

A cross linkage structure is typically formed using cross-linking agents, and it is preferable that a structure is formed by reacting suitable cross-linking agents based on types of cross-linking functional groups of an adhesive polymer so that sufficient cross linking density to increase heat resistance as an adhesive and adhesive deposit prevention effect may be obtained. For example, when a cross-linking functional group utilized in cross-linking is a carboxyl group, it is preferable that a cross linkage structure is formed using bis amide derived cross-linking agents or epoxy resin derived cross-linking agents as cross-linking agents. But a cross linkage structure may also be formed using isocyanate derived cross-linking agents in a range that does not impair effect of the present invention.

Epoxy resin derived cross-linking agents, for example, bisphenol A type epoxy resins, bisphenol F type epoxy resins, cresol novolak type epoxy resins, phenol novolak type epoxy resins, etc. also be used. The epoxy equivalent of epoxy resin derived cross-linking agents is usually 70 to 400, and preferably 80 to 300.

Bis amide derived cross-linking agents, for example, bisaziridine derivatives of dibasic acid, such as iso phthaloyl bis(2-methyl aziridine), etc. may also be used. In addition, since bisamide derived cross-linking agents may react at comparatively low temperature with adhesive polymer having carboxyl groups, it is especially preferable to easily obtain sufficient cross-linking density.

In an adhesive composition of the present invention, in order to increase heat resistance of the adhesive it is preferable that cross-linking component unit is included at 0.01 to 20 mass % in the adhesive composition, and is more preferable that 0.05 to 10 mass %.

In an adhesive polymer used in the present invention tackifiers may also be used with adhesive polymers as in conventional pressure sensitive adhesives.

An adhesive polymer used in the present invention may be prepared by methods known to those skilled in the art. For example, acrylic derived adhesive polymer may be obtained by polymerizing a mixed monomer including (a) (meth)acrylic monomer that has alkyl group having 4 to 8 carbons in molecule, and (b) (meth)acrylic monomer that has carboxyl group in molecule. In this case, copolymerization may be carried out by standard methods of polymerization, such as solution polymerization. Moreover, in order to raise compatibility with a nonadhesive polymer, it is preferable that large amounts of the above described components (a) and (b) are included in all monomers, and specifically it is preferable that the above described components (a) and (b) are included 65 mol % or more in sum total in all monomers, and more preferable that 70 mol % or more.

(2) Nonadhesive Polymer

A glass transition point (Tg₂) of a nonadhesive polymer by a differential scanning calorimeter used for the present invention is −5° C. or less.

If a glass transition point (Tg₂) by a differential scanning calorimeter exceeds −5° C., low-temperature adhesive property, and particularly the pressure sensitivity at a temperature of less than 0° C. will decline, and therefore application to adhesive film used outdoors for an object, such as advertisement, will become difficult. In the present invention, although there is no limitation in lower limit of a glass transition point (Tg₂), when the glass transition point (Tg₂) is too low, there is a possibility that adhesive property (releasing strength and holding power) of adhesive composition obtained may decline. Therefore, a glass transition point (Tg₂) is preferably −70° C. or more.

Moreover, in the present invention, a glass transition point as determined by a differential scanning calorimeter of a nonadhesive polymer (Tg₂) is preferably no more than a glass transition point (Tg₁) as determined by a dynamic viscoelasticity measuring method of an adhesive polymer, and more preferably a Tg difference (Tg₁−Tg₂) that is in a range of 20 to 65° C. Tack suppression effect can be increased easily, almost without disturbing adhesive property including low-temperature adhesive property of the adhesive polymer, if the glass transition point of both polymers is in such a relationship.

Further, a nonadhesive polymer as used in the present invention is a crystalline polymer with a fusing point by a differential scanning calorimeter higher than 25° C.

When a fusing point as determined by a differential scanning calorimeter is 25° C. or less, tackiness is substantially demonstrated at ordinary temperatures and tack of the composition obtained may not be suppressed effectively. In the present invention, there is no particular limitation in an upper limit of a fusing point of a nonadhesive polymer.

However, there is a possibility that compatibility with an adhesive polymer may fall when the fusing point is too high, and it is preferable that the fusing point of the nonadhesive polymer be 100° C. or less.

Furthermore, a nonadhesive polymer in the present invention has compatibility with an adhesive polymer in uncrystallized state.

A nonadhesive polymer having no compatibility with an adhesive polymer will interfere with the low-temperature performance of the adhesive polymer, and, as a result, the low-temperature adhesive property of a composition will fall.

As used in this specification, “compatibility” may be determined by three factors, (1) change of transparency, (2) solution transparency and (3) transmittance of polarized light by a polarization microscope.

(1) Change of Transparency (Haze) of Adhesive Composition

For example, in a film adhesive (film-like adhesive) comprising an adhesive composition used in the present invention, and having a thickness in a range of 20 to 60 μm, a film adhesive is heated at a temperature no less than a fusing point of an nonadhesive polymer and compared with a film adhesive that is heated at a temperature less than the fusing point. In a case of where it is heated at a temperature less than the fusing point, the nonadhesive polymer usually forms a plurality of fine crystals. A phase of the nonadhesive polymer is dispersed in matrix phase including the adhesive polymer. Although, it shows comparatively highly transparency, haze value measured using a color difference meter shows at least 5% (usually 20% or less). On the other hand, at a temperature of the fusing point or more of the nonadhesive polymer, the nonadhesive polymer melts, and so that a state where the nonadhesive polymer and the adhesive polymer are melted mutually is shown to lower the haze value and the film adhesives seem to be almost transparent. Moreover, even if the nonadhesive polymer melts, when the nonadhesive polymer and adhesive polymer do not mutually melt, a haze value hardly changes. In such a case, smaller haze value means better compatibility. Therefore, in the case that the nonadhesive polymer and the adhesive polymer melt mutually, a haze value measured using a color difference meter is preferably 2% or less, and especially preferably 2% or less.

(2) Whether a Solution Including a Nonadhesive Polymer and an Adhesive Polymer is Transparent or Not

In simple, when a first transparent solution including an adhesive polymer dissolved therein, and a second transparent solution including a nonadhesive polymer dissolved therein are mixed, it can be judged by whether resulting mixed liquor is transparent or not.

(3) Transmittance of Polarized Light by a Polarization Microscope

As is known well, if polarization axes of two polarizing plates are made to intersect perpendicularly, light will no longer be transmitted to give almost black view. Thus, a film adhesive comprising an adhesive composition of the present invention is placed between two polarizing plates made to intersect perpendicularly and observation is performed. At ordinary temperature, micro crystal of nonadhesive polymer rotates polarization plane of light coming into the film adhesive, and the light is transmitted through two polarizing plates. Since the direction of crystal axis is usually random, a crystal that works to transmit incident light through two polarizing plates by rotating polarization plane of light by exactly 90 degrees and a crystal through which light cannot transmit may exist at the same time. Finer dispersion of micro crystal of the nonadhesive polymer gives higher compatibility between the nonadhesive polymer and the adhesive polymer. Therefore, finer crystal size displays whole of film half-light within a microscopic field (magnification 100 to 200 times) if compatibility between both of the polymers is high. When the compatibility between both of the polymers is low, the crystal size is large, and therefore crystalline strucutre can be recognized as a bright point dotted on dark background. In addition, in the state where the nonadhesive polymer melts, and the adhesive polymer and nonadhesive polymer melt together, the mixture of these polymers included in the film adhesive is optically isotropic, and gives a dark view compared with a mixture at room temperature.

Non-limiting examples of a nonadhesive polymer used in the present invention has a glass transition point (Tg₂) as determined by a differential scanning calorimeter of −5° C. or less and a fusing point measured by a differential scanning calorimeter being higher than 25° C. Such polymers are compatible with the adhesive polymer in an uncrystallized state, and include but are not limited to polyester polyols, such as poly caprolactones; polyols, such as polycarbonate polyols, or crystalline polyurethanes obtained by polymerizing these polyols and diisocyanate compounds. In addition, these polymers may be used independently or may be used in combination.

Additional nonadhesive polymers used in the present invention include, a polymer that has an alkylene skeleton of 4 to 6 carbon atoms in the molecule with the promise that the polymer has (1) a high degree of crystallinity, and (2) an effective fuming point such that the polymer is not tacky at room temperature (3 is soluble in organic solvents and (4) is compatible with the adhesive polymer. Furthermore, a nonadhesive polymer having poly caprolactone skeleton or polycarbonate skeleton is more preferable. A nonadhesive polymer having a polycarbonate skeleton in molecule is preferable and generally, water resistance and anti-hydrolysis property of an adhesive composition.

Moreover, compatibility with an adhesive polymer having functional groups including OH group in molecule is effectively improved, when a nonadhesive polymer preferably has functional groups (hydroxyl group, carboxyl group, etc.) including OH groups.

Although there are present no limitation about molecular weight, are molecular weight is usually in the range 2,000 to 200,000 as weight average molecular weight, and more preferably in the range of 3,000 to 100,000.

(3) Adhesive Composition

An adhesive composition in the present invention includes the above described adhesive polymer and nonadhesive polymer, wherein tack suppression and low-temperature adhesive property of the adhesive composition are simultaneously attained.

An adhesive composition in the present invention specifically has a probe tack measured by a method based upon ASTM D2979 under conditions of 25° C., a contact pressure of 100 g/cm², a contact time of 1 second, and a releasing speed of 10 mm/sec (hereinafter referred to as “probe tack” simply) that is preferably lower than 7 N, and more preferably is 6N or less, and especially preferably is 5 N or less.

A probe tack in this range provides for easier release of an adhesive sheet and also makes it easier to attain accurate positioning of the adhesive sheet.

In the present invention, although lower limit of a probe tack is not especially limited as long as pressure sensitivity at low temperature is not impaired, it is preferably 2 N or more in view of easy temporary sticking process obtained, and more preferably 3 N or more.

An adhesive composition of the present invention preferably has a 180 degrees releasing strength (hereinafter referred to as simply “−5° C. releasing strength”) of 20 N/25 mm or more measured at a 300 mm/minute releasing speed at −5° C. after being left as it is for 5 minutes while adhering to a melamine baking finish plate at −5° C., more preferably 22 N/25 mm or more, and especially preferably 23 N/25 mm or more.

When the release strength is in the range of −5° C., sufficient pressure sensitivity is demonstrated in an environment at low temperatures less than 0° C., and application to an adhesive sheet used out in fields for the purpose, such as an advertisement.

In the present invention, although an upper limit of a −5° C. release strength is not especially limited, preferably it is 50 N/25 mm or less in view of making tack control easier.

In an adhesive composition in the present invention, it is preferable that a suitable combination of a nonadhesive polymer and an adhesive polymer may suitably be selected so that the above described characteristics, such as tack suppression and low-temperature pressure sensitivity, may be demonstrated.

In a combination of a nonadhesive polymer and an adhesive polymer, for example, (i) a probe tack ratio (TAf/TBf) that is a ratio of a probe tack (TAf) of an adhesive composition including a nonadhesive polymer 10 mass parts to an adhesive polymer 90 mass parts, to a probe tack (TBf) of an adhesive composition including the adhesive polymer concerned independently is less than 0.9, and preferably 0.88 or less, and (ii) a −5° C. releasing strength ratio (PAf/PBf) that is a ratio of a −5° C. releasing strength (PAf) of the adhesive composition including a nonadhesive polymer 10 mass parts to an adhesive polymer 90 mass parts to a −5° C. releasing strength (PBf) of an adhesive composition including the adhesive polymer concerned independently is 0.7 or more, and preferably 0.8 or more.

In a combination in which a probe tack ratio (TAf/TBf) exceeds 0.9, tack suppression effect at normal temperature may not necessarily be enough, and accurate positioning of the site for sticking of an adhesive sheet may become difficult. On the other hand, in a combination in which a −5° C. releasing strength ratio (PAf/PBf) becomes less than 0.7, low-temperature adhesiveness of a composition, especially pressure sensitivity at a temperature less than 0° C. in an environment at low temperature falls, and sometimes sticking of an adhesive sheet may become difficult.

In an adhesive composition in the present invention, it is preferable that an adhesive polymer and a nonadhesive polymer are present in a ratio so that effective tack suppression and low-temperature press fitting nature may be demonstrated.

It is preferable that an adhesive polymer is included 70 to 97 mass % in an adhesive composition, it is more preferable 75 to 95 mass %, and it is especially preferable 80 to 94 mass %. There is a possibility that a pressure sensitivity, especially pressure sensitivity at low temperature may fall when a content rate of an adhesive polymer is less than 70 mass %, on the other hand if it exceeds 97 mass %, there is a possibility that a normal temperature tack may not be controlled low.

The nonadhesive polymer is preferably included 2 to 29 mass % in an adhesive composition, more preferably 4 to 24 mass %, and especially preferably 5 to 19 mass %.

When a content of a nonadhesive polymer is less than 2 mass %, there is a possibility that a normal temperature tack may not be controlled at low temperatures and when it exceeds 29 mass % on the contrary, there is a possibility that pressure sensitivity may fall.

In an adhesive composition of the present invention, a first solution with the above described adhesive polymer dissolved therein, and a second solution with a nonadhesive polymer dissolved therein are mixed to obtain a mixed solution with the adhesive polymer and the nonadhesive polymer almost uniformly dispersed therein. Subsequently, this mixed solution is preferably dried to obtain the adhesive composition.

In an adhesive composition obtained by such a manufacturing method, when an adhesive polymer and a nonadhesive polymer form mutual phase separated structure at a temperature less than a fusing point of a nonadhesive polymer, a phase separated structure where phase including large amount of the nonadhesive polymer is finely and uniformly dispersed is obtained, and thereby a nonadhesive polymer may exist almost without blocking adhesive property (low-temperature releasing strength etc.) of an adhesive polymer, and tack control effect is easily improved.

Preferably, the adhesive polymer and nonadhesive polymer can be dissolved low molecular organic solvent having 4 to 8 carbons that has alkyl groups conjugating with benzene ring or carbonyl group in a molecule. Such organic solvents included but are not limited to for example, methyl ethyl ketone, ethyl acetate, toluene, etc.

Mixing process of a first solution and a second solution may be performed by conventional methods using mixing equipments, such as a homogeneous mixer and a planetary mixer, and thus each polymer may be uniformly dissolved or dispersed.

Drying process of mixed solution obtained may usually be performed at temperatures of 60 to 180° C. for dozens of seconds to several minutes.

In addition, when an adhesive composition is a cross-linkable polymer, a third solution containing a cross-linking component is added to a mixed solution of the first solution and the second solution, and then all solutions are mixed together uniformly.

Other additives may be included, provided the additives do not diminish the effectiveness of the adhesive composition. If such additives are used, they are present in amount consistent with the use as known to those skilled in the art. Such additives include but are not limited to viscosity regulators, defoaming agents, leveling agents, UV absorbents, antioxidants, pigments, anti-mold agent, elastic minute balls comprising an adhesive polymer or nonadhesive rubber derived polymer, tackifiers, catalysts promoting cross-linking reaction, etc.

2. Low Temperature Applicable Adhesive Sheet

A low temperature applicable adhesive sheet in the present invention comprises a substrate and an adhesive layer made of an adhesive composition and such adhesive composition is disposed on at least one of major surface of the substrate.

Substrates used in the present invention, include for example, a substrate made from paper, coated paper, metal films, and polymer films. Substrates comprising a polymer film are preferred.

Polymer films comprising one or more synthetic polymer including at least one kind selected from a group of non-crystalline polyesters, plasticized polyesters, polyurethanes, polyolefins, and ethylene-acrylate copolymers etc. are preferable. Furthermore, polyolefins, especially ionomers, or ethylene-vinyl acetate copolymer modified polyolefins are more preferable.

A substrate used in the adhesive sheet of the present invention preferably has 50% or more of low-temperature elongation at −5° C. and by elastic stress rate of 300 mm/minute, more preferably 60% or more, and especially preferably 70% or more.

In the present invention, although there is no particular limitation about the upper limit of the above described low-temperature elongation, as long as there is no possibility that a wrinkle may occur on a sheet, or a sheet may fracture by a hand work at ordinary temperature, it is preferable that the above described low-temperature elongation is 1,000% or less.

In addition, “low-temperature elongation” represents a ratio of an elongation of the substrate at breaking by strain to a length of a strain direction of a substrate before straining.

It is preferable that a substrate used in an adhesive sheet of the present invention further has 50% elongation stress of 10 to 200 MPa at −5° C. and in 300 mm elastic stress rate, it is more preferable that 12 to 150 MPa, and it is especially preferable that 15 to 100 MPa.

If the above described 50% elongation stress is 200 MPa or more, there is a possibility that neither irregularity of an adherend nor curve may not be followed at a low temperature of −5° C., and on the other hand, if the above described 50% elongation stress is less than 10 MPa, the elongation of a substrate will become excessively large, and there is a possibility that wrinkle may be given on a sheet or a sheet may fracture by an ordinary temperature hand work. “50% elongation stress” represents a stress when a substrate is elongated by 50%.

In the present invention, although there is no particular limitation about a thickness of a substrate, suitable thickness is generally based on the application and such thickness is generally 5 to 500 μm, and preferably 10 to 300 μm.

Furthermore, a substrate used in an adhesive sheet of the present invention may transmit visible light and ultraviolet radiation, and may also be a substrate reflecting light, such as a retroreflection sheet. It may be colored, or the sheet may contain one or more images and such an adhesive sheet may be used as an ornamental sheet or a marking film.

Although a substrate used in an adhesive sheet of the present invention may include an adhesive layer on at least one main surface, a liner may also be included on at least one main surface of the substrate, and the adhesive layer may be indirectly prepared via this liner.

A liner having paper or plastics film as principal component may be included. The paper liner usually has releasing coatings (releasing layer), such as a polyethylene coat and a silicone coat on a surface of paper. When the releasing coating of a silicone coat is laminated, it is typical that under coats, such as clay coat and a polyethylene coat, are present between the paper and the releasing coating layer.

Liners with fine irregularity formed on the stripping face are preferable as liners used in the present invention. In an adhesive sheet with such a liner formed thereon, irregularity transferred from the irregularity of a liner stripping face can be prepared on a surface (adhesion face) of an adhesive layer prepared on a liner. Furthermore, minute glass beads etc. can be disposed in a predetermined position of an adhesive layer by giving a desired pattern to the irregularity of a liner releasing face. And, after the minute glass beads have been disposed on the liner, an adhesive layer may be formed on the liner and the minute glass beads will be diffused onto the adhesive layer.

In addition, irregularity of a liner stripping face may be arranged with regularly repeated pattern, and may be irregularity having a rough surface with irregular pattern.

When a first solution containing an adhesive polymer is in dissolved state and a second solution similarly including a nonadhesive polymer is in dissolved state are mixed together, the mixed solution is applied on a substrate or a liner. This mixture is dried to form an adhesive layer on the liner. In addition, when adding a cross-linking component, predetermined quantity of a third solution comprising a cross-linking component may be added to the mixed solution obtained by mixing first solution and the second solution.

Moreover, as solvents dissolving an adhesive polymer and a nonadhesive polymer, a solvent dissolving each polymer is preferably a comparatively low molecular organic solvent having 4 to 8 carbons that has benzene ring or carbonyl group, and alkyl group conjugating to the benzene ring or the carbonyl group in a molecule so that it may have a good compatibility to both of an adhesive polymer and a nonadhesive polymer used in an adhesive composition of the present invention and an adhesive polymer and a nonadhesive polymer can be uniformly dissolved in a mixed solution.

Moreover, as such organic solvents, for example, methyl ethyl ketone, ethyl acetate, toluene, etc. may be mentioned.

When such an organic solvent is used, an adhesive polymer has one or more alkyl groups having 4 to 8 carbons, and one or more carboxyl groups in a molecule, a percentage of a monomer unit comprising the alkyl groups having 4 to 8 carbons included in the adhesive polymer molecule is 60 to 99 mol %, and it is preferable that the one or more alkyl groups having 4 to 8 carbons always include a butyl group, and that a nonadhesive polymer has alkylene skeleton having 4 to 6 carbons in a molecule. In an applied film formed from a coating material by such combination, an adhesive polymer and a nonadhesive polymer have uniform and minute phase separated structure, and thereby a nonadhesive polymer may exist almost without blocking adhesive property of an adhesive polymer, and tack control effect is easily improved in an adhesive composition comprising such an applied film.

A coating material that forms an adhesive layer can be formed by dissolving or dispersing each material uniformly using mixing equipments, such as a homogeneous mixer and a planetary mixer. Drying process at a time of forming an adhesive layer is usually performed at a temperature of 60 to 180° C. Drying time is usually dozens of seconds to several minutes. An adhesive layer usually has a thickness of 5 to 1,000 μm, preferably 10 to 500 μm, and especially preferably 15 to 100 μm. Well-known means, such as a knife coater, a roll coater, a die-coater, and a bar coater, may be used for an application means. In addition, a nonvolatile component in a first solution, a second solution, and a mixed solution that mixes these solutions approximately in a uniform state, is preferably 10 to 70 mass %.

As adherend to which an adhesive sheet of the present invention is stuck, materials having adhering face formed by (1) metals, such as aluminum, stainless steel, steel, and zinc steel plate; (2) resins, such as polyimides, acrylate resins, polyurethanes, melamine resins, epoxy resins, and vinyl chlorides; (3) inorganic oxide materials, such as ceramics and glass etc. may be used. Moreover, materials having a coated face as an adhering face may be used.

### EXAMPLES

Examples of the present invention will be described hereinafter.

A glass transition temperature (Tg₂) and a fusing (or melting) point (Tm) of a nonadhesive polymer of each Example and Comparative example were specified from endotherm peak temperature of the obtained chart, by measuring a quantity of heat, using a differential scanning calorimeter (type number) DSC-2CC manufactured by PerkinElmer, Inc., in a temperature range of −60° C. to 180° C., at temperature rising velocity for 10° C./minute.

When measuring a glass transition temperature (Tg₁) of an adhesive polymer, an adhesive polymer solution was applied on a release coated paper, and the dried film (about 30 μm in thickness) was cylindrically rounded to be used as a specimen. An elastic loss (tanδ) was measured using a dynamic viscoelasticity spectrum meter (manufactured by Rheometric Scientific F. E. Ltd., type number: RDA-H) under the conditions of a temperature region of −60 to 200° C., the share mode in torsion mode, and the frequency of one rad/s to give a glass transition temperature (Tg₁).

### Example 1

As a nonadhesive polymer, a polycaprolactone having a carboxyl group (manufactured by DAICEL CHEMICAL INDUSTRIES, LTD., Placcel (TM) item number 220BA, Mw=4,900 by GPC measurement, Tg₂=−60° C., fusing point=55° C. (abbreviated to “NTP1” hereafter)) was used. This nonadhesive polymer was dissolved in toluene and a nonadhesive polymer solution having nonvolatile matter concentration of 20 mass % was prepared.

Next, a mixed monomer containing butyl acrylate 50 mass parts, 2-ethyl hexyl acrylate 33 mass parts, methyl acrylate 10 mass parts, and acrylic acid 7 mass parts was polymerized in solution in a mixed organic solvent of toluene and ethyl acetate (mass ratio 3:7) to give an adhesive polymer (may be abbreviated to “SAP 1” hereafter), and thus an adhesive polymer solution having solid content concentration of 30 mass % was prepared. A glass transition temperature by a dynamic viscoelasticity method of this adhesive polymer was −22° C., and a weight average molecular weight (Mw) by GPC measurement was 480,000.

Next, the nonadhesive polymer (NTP1) solution and adhesive polymer (SAP1) solution prepared as described above, respectively, were mixed and stirred so that a mass ratio of a nonvolatile matter may be set to 90:10. As a cross-linking agent, 0.2 mass parts iso-phthaloyl-bis(2-methyl aziridine) was added to the obtained mixed solution 100 mass parts and stirred, and the adhesive composition solution was obtained. Obtained adhesive composition solution was transparent.

Next, on the releasing face of a liner, the obtained adhesive composition solution was applied using a knife coater, dried under a condition at 90° C. and for 5 minutes, and the adhesive layer comprising an adhesive composition having a thickness of 35 μm was formed on the liner. Subsequently, a substrate having a thickness of 80 μm and comprising ethylene vinyl acetate copolymer modified polyolefin film was dry-laminated onto the adhesive layer to obtain an adhesive sheet having an adhesive layer at one side.

In addition, in the used substrate, the low-temperature elongation measured under a condition at −5° C. and 300 mm/minute of elastic stress rate showed 200%, and 50% elongation stress measured under a same condition at −5° C. showed 21 MPa. Evaluation results of the obtained adhesive sheet are collectively shown in Table 1.

### Example 2

As a nonadhesive polymer, a poly carbonate diol (manufactured by DAICEL CHEMICAL INDUSTRIES, LTD., Placcel (TM) item number CD220, Mw=7,300 by GPC measurement, Tg₂=−60° C., fusing point=53° C. (abbreviated to “NTP2” hereafter)) was used. This nonadhesive polymer was dissolved in toluene and a nonadhesive polymer solution having nonvolatile matter concentration of 20 mass % was prepared.

Next, a mixed monomer containing butyl acrylate 93 mass parts, acrylnitrile 3 mass parts, and acrylic acid 4 mass parts was polymerized in solution in a mixed organic solvent of toluene and ethyl acetate (mass ratio 3:7) to give an adhesive polymer (may be abbreviated to “SAP2” hereafter), and thus an adhesive polymer (SAP2) solution having solid content concentration of 30 mass % was prepared. A glass transition temperature by a dynamic viscoelasticity method of this adhesive polymer was −21° C., and a weight average molecular weight (Mw) by GPC measurement was 590,000.

In other conditions, the same method as Example 1 was used, and an adhesive sheet was manufactured. Evaluation results of the obtained adhesive sheet are collectively shown in Table 1.

### Example 3

As a nonadhesive polymer, the polycarbonate diol used in Example 2 and isophorone diisocyanate was polymerized in toluene to prepare a crystalline polyurethane (Mw=15,000 by GPC measurement, Mw/Mn=2.8, Tg₂=−60° C., a fusing point=49° C. (may be abbreviate to “NTP3” hereafter)). Except that this polyurethane was used as a nonadhesive polymer (NTP3) solution, an adhesive sheet was manufactured as in Example 1. In addition, a nonvolatile matter concentration of this solution showed 20 mass %. Evaluation results of the obtained adhesive sheet are collectively shown in Table 1.

### Example 4

An adhesive sheet was manufactured as in Example 1, except that as a nonadhesive polymer, a polycaprolactone (manufactured by DAICEL CHEMICAL INDUSTRIES, LTD., Placcel (TM) item number 220, Mw=5,700 by GPC measurement, Mw/Mn=2.0, Tg₂=−60° C., fusing point=59° C. (abbreviated to “NTP4” hereafter)) was used, and this nonadhesive polymer (NTP4) was dissolved in toluene and a nonadhesive polymer solution having nonvolatile matter concentration of 20 mass % was prepared. Evaluation results of the obtained adhesive sheet are collectively shown in Table 1.

### Comparative Example 1

An adhesive sheet was manufactured as in Example 1, except that as a nonadhesive polymer, a non-crystallized polymer (manufactured by Hercules Incorporated, Hydrogenated pentaerythritol resin ester, Foral (TM), item number 105, Mw=970, Mw/Mn=1.1, Tg₂=−57° C., (may be abbreviated to “NTP5” hereafter)) was used, and this nonadhesive polymer was dissolved in mathyl ethyl ketone and a nonadhesive polymer solution having nonvolatile matter concentration of 20 mass % was prepared. Evaluation results of the obtained adhesive sheet are collectively shown in Table 1.

### Reference Examples 1 and 2

In order to show adhesive properties of each of the independent adhesive polymer used in each Example and Comparative example, adhesive sheets were prepared as in Example 1 and Example 2, respectively, except having not used the nonadhesive polymer solution. Evaluation results of the obtained adhesive sheet are collectively shown in Table 1.

For reference, in addition, for, a glass transition temperature (Tg₂) of the adhesive polymers (NTP1 and NTP2) used in Example 1 and Example 2 was measured from endotherm peak temperature of the obtained chart, by measuring a quantity of heat, using a differential scanning calorimeter (type number) DSC-2CC manufactured by PerkinElmer, Inc., in a temperature range of −60° C. to 180° C., at temperature rising velocity for 10° C./minute, and the adhesive polymer (NTP1) used in Example 1 showed −29° C., and the adhesive polymer (NTP2) used in Example 2 showed −36° C.

(Evaluation Method)

Adhesive sheets obtained in each Example, Comparative example, and Reference example were estimated by following test methods. Evaluation results are collectively shown in Table 1.

(1) Crystallinity

Samples were observed at 25° C. using a polarization microscope, and a sample in which crystal was confirmed was given “observed”, and a sample in which no crystal was confirmed was given “not observed”.

(2) 20° C. Releasing Strength

The adhesive sheets obtained in each Example, Comparative example, and Reference example were cut by 200 mm×25 mm, ant the cut sheet was stuck to a melamine baking finish plate by PALTEC Co., Ltd. as an adherend under 20° C. environment using a press fitting roller to prepare a test specimen. Adhesion of a sheet was performed by a method according to JIS Z0237 8.2.3. The releasing strength in a direction of 180 degrees at 300 mm/minute releasing speed was measured using a Tensilon after kept standing for 48 hours under a same temperature after completion of adhesion.

(3)-5° C. Releasing Strength

The adhesive sheets obtained in each Example, Comparative example, and Reference example were cut by 200 mm×25 mm, ant the cut sheet was stuck to a melamine baking finish plate by PALTEC Co., Ltd. as an adherend under −5° C. environment using a press fitting roller to prepare a test specimen. Adhesion of a sheet was performed by a method according to JIS Z0237 8.2.3. The releasing strength in a direction of 180 degrees at 300 mm/minute releasing speed was measured using a Tensilon after kept standing for 5 minutes under a same temperature after completion of adhesion.

(4) Probe Tack

Based on ASTM D2979, measured using a probe tack tester with a thermostat manufactured by TESTER SANGYO CO., LTD. Measurement conditions were temperature of 25° C., contact pressure 100 g/cm², contact time 1 second, and releasing speed 10 mm/second. In addition, as evaluation values, an average by five measurements (N=5) was used.

(5) The Rate of Tack Attenuation

Calculated by a relational expression shown below.

Rate of tack attenuation={1−[probe tack of composition comprising nonadhesive polymer]÷[probe tack of adhesive polymer itself]}×100.

TABLE 1

| | | | | | −5° C. | | |
|---|---|---|---|---|---|---|---|
| | | Crystallinity of | | 20° C. releasing | releasing | | |
| | Nonadhesive | nonadhesive | Adhesive | strength | strength | Probe tack | Rate of tuck |
| | polymer | polymer | polymer | [N/25 mm] | [N/25 mm] | [N] | attenuation |
| Example 1 | NTP1 | Observed | SAP1 | 21 | 36 | 4.3 | 22% |
| Example 2 | NTP2 | Observed | SAP2 | 13 | 25 | 3.6 | 16% |
| Example 3 | NTP3 | Observed | SAP1 | 20 | 35 | 4.8 | 13% |
| Example 4 | NTP4 | Observed | SAP1 | 18 | 36 | 3.9 | 29% |
| Reference | Not observed | — | SAP1 | 24 | 37 | 5.5 | 0% |
| example 1 | | | | | | | |
| Reference | Not observed | — | SAP2 | 14 | 31 | 4.3 | 0% |
| example 2 | | | | | | | |
| Comparative | NTP5 | Not observed | SAP1 | 21 | 17 | 5.4 | 2% |
| example 1 | | | | | | | |

### EVALUATION

In adhesive sheets in Examples 1 to 4, excellent pressure sensitive adhesion was possible in a temperature range of 20 to −5° C. Moreover, low tack was given, and outstanding positioning property and outstanding re-sticking performance were demonstrated compared to a case where an adhesive polymer was used independently (reference example). And, sticky touch was also mitigated and it was able to respond easily also to quick handwork by the expert. On the other hand, in the adhesive sheet by Comparative example 1, although excellent pressure sensitive adhesion was possible at 20° C., −5° C. pressure sensitivity was low, and was estimated that low-temperature applicability was low as compared to samples in Example. And, tack suppression effect by addition of a nonadhesive polymer was not observed, and positioning property and re-sticking performance have not improved.

As described above, according to the present invention, there is provided an adhesive composition which enables to effectively enhance pressure sensitive adhesivity at low temperatures and tack suppression effect at the same time, which makes easy an accurate positioning of the site for sticking, and which can be easily stuck by press fitting even in an environment of low temperature of lower than 0° C. and an adhesive sheet constituted with the composition as an adhesive layer.

What is claimed is:

1. An adhesive composition having pressure sensitivity comprising:
an adhesive polymer and
a nonadhesive polymer,
wherein the adhesive polymer has a glass transition point by a dynamic viscoelasticity measuring method of −60° C. to −5° C., and the nonadhesive polymer has a glass transition point by a differential scanning calorimeter of −5° C. or less, and a fusing point of higher than 25° C. by a differential scanning calorimeter, and also has compatibility with the adhesive polymer in uncrystallized state.

2. An adhesive composition according to claim 1, wherein the adhesive composition is obtained by mixing almost uniformly a first solution in which the adhesive polymer is dissolved, and a second solution in which the nonadhesive polymer is dissolved to give a mixed solution, and drying the mixed solution.

3. An adhesive composition according to claim 1, wherein a probe tack measured by a method based on ASTM D2979 under conditions of 25° C., contact pressure of 100 g/cm², contact time of 1 second, and releasing speed of 10 mm/second is lower than 7N, and a 180 degrees releasing strength measured at a releasing speed of 300 mm/minute, in −5° C. environment is 20 N/25 mm or more, after being stuck to a melamine baking finish plate and being left to stand for 5 minutes under −5° C. environment.

4. An adhesive composition according to claim 1, wherein the adhesive polymer has at least one alkyl group having 4 to 8 carbons including at least one butyl group, and at least one carboxyl group, and monomer units including alkyl groups having 4 to 8 carbons in a percentage of 60 to 99 mole % in all molecules.

5. An adhesive composition according to claim 1, wherein the adhesive polymer has a polycaprolactone skeleton or a polycarbonate skeleton in the molecule.

6. A low temperature applicable adhesive sheet comprising:
a substrate, and
an adhesive layer comprising the adhesive composition arranged at least on one main surface of the substrate,
wherein the adhesive composition includes an adhesive polymer and a nonadhesive polymer, the adhesive polymer has a glass transition point by a dynamic viscoelasticity measuring method of −60° C. to −5° C., and the nonadhesive polymer has a glass transition point by a differential scanning calorimeter of −5° C. or less, and a fusing point of higher than 25° C. by a differential scanning calorimeter, and also has compatibility with the adhesive polymer in uncrystallized state, and
the substrate includes a polymer film, and has a breaking elongation of 50% or more at −5° C. at an elastic stress rate of 300 mm/min and a 50% elongation stress of 10 to 200 MPa at an elastic stress rate of 300 mm/min at −5° C.
